(19) United States
(12) Patent Application Publication
(10) Pub. No.: US 2023/0321187 A1
(43) Pub. Date: Oct. 12, 2023

(54) OCTOPUS-DERIVED PEPTIDE OCTOPROMYCIN HAVING ANTIBACTERIAL AND ANTI-BIOFILM ACTIVITY AGAINST ACINETOBACTER BAUMANNII

(71) Applicant: NATIONAL MARINE BIODIVERSITY INSTITUTE OF KOREA, Seocheon-gun, Chungcheongnam-do (KR)

(72) Inventors: Il Son Whang, Seocheon-gun, Chungcheongnam-do (KR); Mahanama Dezoysa, Yuseong-gu, Daejeon (KR); Pathiranage Dinusha Cathurangi Rajapaksha, Yuseong-gu, Daejeon (KR); Chamilani Lalanthi Nikapitiya, Yuseong-gu, Daejeon (KR)

(21) Appl. No.: 18/042,238

(22) PCT Filed: Aug. 20, 2021
(86) PCT No.: PCT/KR2021/011092

§ 371 Date: Feb. 20, 2023

(30) Foreign Application Priority Data
Aug. 20, 2020 (KR) 10-2020-0104723

(51) Int. Cl. A61K 38/17; A61P 31/04

(52) U.S. Cl. CPC A61K 38/1767; A61P 31/04

(57) ABSTRACT

The present invention relates to octopromycin, which is a synthetic peptide having excellent antibacterial activity against Acinetobacter sp. bacteria, and an antibacterial composition comprising the octopromycin as an active ingredient. Octopromycin or a fragment thereof according to the present invention exhibits high inhibitory effects on the growth and biofilm formation of Acinetobacter baumannii, which is a multi-drug resistant bacterium causing nosocomial infection, in addition to being free of cytotoxicity, thus finding advantageous applications in treating and preventing the infection of Acinetobacter baumannii.

### TECHNICAL FIELD

The present invention relates to octopromycin, which is a synthetic peptide having excellent antibacterial activity against Acinetobacter sp. bacteria, which is multi-drug resistant, and a use thereof.

### BACKGROUND ART

In the development of anti-infective agents, the continuous emergence of antibiotic-resistant bacteria has become a major issue. Antimicrobial peptides (AMPs) are considered an important part of host defense peptides to be resistant to bacterial infection as well as an alternative to antibiotics. Moreover, the AMP is a multi-functional substance on skin and mucosal surfaces that exhibits direct antibacterial activity against various bacteria, viruses, fungi, etc. (Gordon et al, Current eye research, 30:505, 2005). Most AMPs have low molecular weights (<10 kDa), and are further effective in antibacterial activity against pathogens due to unique properties of positively charged ions, amphiphilicity, and α-helical structures. Membrane permeability is the best known mechanism to explain the actions of cationic AMPs (Lei et al, American journal of translational, 11: 3919, 2019). The AMPs have been discovered in almost all organisms and display remarkable functional and structural diversity. In addition, since the AMPs have immunomodulatory properties in addition to direct antimicrobial activity, the AMPs have the potential to be developed as novel therapeutic agents (Mahlapuu et al, Frontiers in cellular and infection microbiology, 6:194, 2016). Several types of AMPs are already on the market or undergoing clinical trials, which provide reasons for the introduction of novel AMP-based drugs in many fields (Pfalzgraff et al, Frontiers in pharmacology, 9:281, 2018).

Marine organisms are rich sources capable of searching for molecules with antimicrobial activity, but AMPs derived from the marine organisms are not well studied. Recently, various AMPs have been isolated from marine organisms such as algae, sponges, crustaceans, and fish. Multi-drug resistance to gram-negative and gram-positive bacteria has been reported in Astacidins 1 and 2 (rich in proline and arginine) derived from crayfish (P. leniusculus) (Lee et al, Journal of Biological Chemistry, 278(10), 7927, 2003): Callinectin AMP (rich in proline, arginine and cysteine) derived from Blue crab (Callinectes sapidu) (Noga et al, Developmental & Comparative Immunology, 35(4), 409, 2011), and Arasin 1 AMP (rich in proline and arginine) derived from spider crab (Hyas araneus) (Stensvag K et al, Developmental & Comparative Immunology, 32(3), 275, 2008).

Octopus belongs to Octopoda and is a potential marine species that may be used in the development of novel drugs due to antibacterial activity, and extracts of Octopus dolfusii and Octopus aegina (Monolisha et al, International Journal of Pharmaceutical Sciences and Research, 4: 3582, 2013) showed antibacterial activity against Vibrio parahaemolyticus, which causes sepsis. In addition, Octominin, which is a novel synthetic peptide derived from defense protein 3 of Octopus minor, exhibits anticandida activity, so that it is confirmed that Octopus minor is a potential marine invertebrate capable of screening for novel AMPs (Nikapitiya et al, Marine Drugs, 18:56, 2020).

ESKAPE pathogens (Enterococcus faecium, Staphylococcus aureus, Klebsiella pneumoniae, Acinetobacter baumannii, Pseudomonas aeruginosa, and Enterobacter spp.) are the leading causes of nosocomial infections (Marturano et al, In Open Forum Infectious Diseases (Vol. 6, No. 12, p. ofz503). US: Oxford University Press., 2019). Over the last three decades, Acinetobater infection has become a major cause of nosocomial infections worldwide. Among them, the most clinically significant pathogen is Acinetobacter baumannii, which has rapidly developed antimicrobial resistance, and some strains have the ability to survive on surfaces of hospital facilities and equipment for a long time (Alsan et al, Journal of clinical outcomes management: JCOM, 17:363, 2010). Most strains of A. baumannii are resistant to antibiotic types such as first- and second-generation cephalosporins, aminopenicillins, and chloramphenicols, and multidrug resistance (MDR) phenotypes rapidly emerge as a result of the ability to both accept exogenous genetic materials and overexpress endogenous resistant genes (Raible et al, Annals of clinical microbiology and antimicrobials, 16: 75, 2017). Therefore, attempts have been made to develop novel peptides to solve the MDR of A. baumannii.

Accordingly, the present inventors made intensive efforts to develop novel AMPs to solve multi-drug resistance of A. baumannii, and as a result, designed and synthesized novel AMPs with characteristic properties using an amino acid sequence of a proline-rich protein 5 gene derived from Octopus minor, and then identified that when the synthesized novel peptides were treated with Acinetobacter baumannii, which is a multi-drug resistant bacterium, the cell growth and biofilm formation were inhibited and the formed biofilm was eradicated even without toxicity, and then completed the present invention.

### DISCLOSURE

### Technical Problem

An purpose of the present invention is to provide a novel antimicrobial peptide (AMP) having an antibacterial effect on multi-drug resistant bacteria.

Another purpose object of the present invention is to provide an antibacterial composition containing the AMP as an active ingredient.

Yet another purpose of the present invention is to provide a pharmaceutical composition for preventing or treating Acinetobacter infection containing the AMP as an active ingredient.

Still another purpose of the present invention is to provide a composition for inhibiting biofilm formation containing the AMP as an active ingredient.

Still yet another purpose of the present invention is to provide a method for treating diseases caused by Acinetobacter infection, including administering the AMP to a subject in need thereof.

### Technical Solution

One aspect of the present invention provides an antimicrobial peptide having a sequence of 5 or more consecutive amino acids in an amino acid sequence represented by SEQ ID NO: 1:

(SEQ ID NO: 1)
¹RRLIRTDTGPIIYDYFKDQLLKKGMVILRESMKNLKGM³⁸.

Another aspect of the present invention provides an antibacterial composition containing the antimicrobial peptide as an active ingredient.

Yet another aspect of the present invention provides a pharmaceutical composition for preventing or treating Acinetobacter infection containing the antimicrobial peptide as an active ingredient.

Still another aspect of the present invention provides a composition for inhibiting biofilm formation containing the antimicrobial peptide as an active ingredient.

Still yet another aspect of the present invention provides a gene encoding the antimicrobial peptide.

Still yet another aspect of the present invention provides a recombinant vector including the gene.

Still yet another aspect of the present invention provides a host cell transformed with the gene or the recombinant vector and a method for producing an antimicrobial peptide using the same.

Still yet another aspect of the present invention provides a method for treating diseases caused by Acinetobacter infection, including administering the antimicrobial peptide to a subject in need thereof.

### Advantageous Effects

According to the present invention, octopromycin or a fragment thereof exhibits high inhibitory effects on the growth and biofilm formation of Acinetobacter baumannii, which is a multi-drug resistant bacterium causing nosocomial infection, in addition to being free of cytotoxicity, and thus can be usefully used for treating and preventing the infection of Acinetobacter baumannii.

### DESCRIPTION OF DRAWINGS

FIG. 1 illustrates results of predicting secondary and tertiary structures of octopromycin according to the present invention. FIG. 1A illustrates a spiral wheel diagram of the alignment of amino acids belonging to a peptide sequence of octopromycin. The number of residues is counted from an amino (N) terminal of the peptide, basic residues are shown in red, and acidic residues are shown in blue. Polar (uncharged) residues are shown in green triangles, and non-polar residues are shown in yellow. FIG. 1B illustrates a three-dimensional structure of octopromycin with positively charged amino acid residues.

FIG. 2 illustrates results of confirming an effect of octopromycin on the growth of A. baumannii. FIG. 2A illustrates time-kill kinetics of octopromycin for the growth of A. baumannii, in which growth inhibition was determined by measuring OD values of a culture solution of A. baumannii treated with 0 to 200 μg/mL of octopromycin at 595 nm. FIG. 2B illustrates a result of confirming the cell viability by performing MTT assay at 24 hours after octopromycin treatment.

Compared with a control (untreated) group, *p≤0.05 was indicated. Error bars represent mean±standard deviation (n=3).

FIG. 3 illustrates results of confirming an effect of octopromycin on morphological and structural changes of A. baumannii by FE-SEM analysis: (A) untreated A. baumannii; (B) A. baumannii treated with octopromycin at MIC (50 μg/mL); (C) A. badman treated with octopromycin at MBC (200 μg/mL); and (D) A. baumannii treated with chloramphenicol (10 μg/mL) as a positive control group.

FIG. 4 illustrates results of confirming an effect of octopromycin on the membrane permeability of A. baumannii and illustrates results confirmed by confocal laser scanning microscopy (CLSM), and illustrates fluorescent images showing cell membrane permeability by propidium iodide (PI) and fluorescein diacetate (FDA) staining in A. baumannii cells treated with octopromycin for 9 hours at 25° C. (A to D) untreated control groups; (E to H) groups treated with octopromycin at MIC (50 μg/mL); (I to L) groups treated with octopromycin at MBC (200 μg/mL); and (M to P) groups treated with chloramphenicol (10 μg/mL) as positive control group.

FIG. 5 illustrates a CLSM result showing a result of confirming an effect of octopromycin on ROS production of A. baumannii, and illustrates fluorescent images and merge images showing ROS production of A. baumannii treated with octopromycin. (A to C) untreated control groups; (D to F) groups treated with octopromycin at MIC (50 μg/mL); (G to I) groups treated with octopromycin at MBC (200 μg/mL); and (J to L) groups treated with chloramphenicol (10 μg/mL) as positive control group.

FIG. 6 illustrates a result of confirming biofilm formation inhibition and biofilm eradication effects of octopromycin on A. baumannii, and illustrates results of using chloramphenicol (10 μg/mL) and treating octopromycin at MIC (50 μg/mL) and MBC (200 μg/mL) as a positive control group. FIG. 6A illustrates biofilm formation inhibition percents, and FIG. 6B illustrates eradication percents of an already formed biofilm. A-1; Biofilm formation inhibition percents in treated and control groups. A-2; Crystal violet staining images for biofilm formation inhibition in treated and control groups. B-1; Biofilm eradication percents of treated and control groups. B-2; Crystal violet staining images of eradicated biofilms in treated and control groups. * P≤0.05 compared to control group. Error bars represent mean±standard deviation (n=3).

FIG. 7 illustrates results of confirming the hemolytic activity of octopromycin on mouse red blood cells, in which FIG. 7A is a visual image showing hemolysis states in supernatants of red blood cells treated with octopromycin, and FIG. 7B is a graph showing hemolysis percents of red blood cells treated with octopromycin. *P≤0.05 compared to (+ye) control group. Error bars represent mean±standard deviation (n=3).

FIGS. 8A and 8B illustrate results of confirming in vitro and in vivo toxicity effects of octopromycin. (A) in FIG. 8A shows cell viability of HEK293 cells treated with octopromycin at various concentrations (0 to 800 μg/mL), and (B) in

FIG. 8A shows cell viability of Raw 264.7 cells treated with octopromycin at various concentrations (0 to 800 μg/mL). (A) in FIG. 8B shows mortality of zebrafish embryos after exposure to octopromycin at various concentrations (0 to 8 μg/mL), and (B) in FIG. 8B shows microscopic images of an unexposed control group and an embryo exposed to octopromycin. Data are expressed as mean±standard deviation (n=3). Magnification: ×2.5.

FIGS. 9A and 9B show results of confirming effects of octopromycin on A. baumannii-infected zebrafish. (A) in FIG. 9A is a schematic diagram showing an experimental process, (B) in FIG. 9A illustrates a result of collecting survival data in the kidney after 48 hours post infection (hpi) after injecting A. baumannii and/or octopromycin intraperitoneally into zebrafish, and (C) in FIG. 9A illustrates results of survival data in the spleen. (A) in FIG. 9B illustrates relative viability of zebrafish after A. baumannii infection and octopromycin treatment. The n number in each group of a control group (PBS), an A. baumannii-infected group, and an octopromycin-treated group was 24, and a representative photograph of each group is shown in (B) in FIG. 9B. *p≤0.05, **P<0.01, ***P<0.001. Error bars represent mean±standard deviation (n=3).

FIG. 10 illustrates results of histological observation of A. baumannii-infected zebrafish. (a) control kidney; (b) degenerated and dilated tubules (DT) and increased cell spaces (black arrow) in A. baumannii-infected kidneys; (c) undamaged DT and few cell spaces in A. baumannii and octopromycin-treated kidney; (d) control spleen; (e) the spleen of A. baumannii-infected group showed extensive hemorrhage (H) and increased density of red pulp (RP); (f) the spleen of A. baumannii and octopromycin-treated group had well-distributed red pulp and white pulp without hemorrhage; (g) control gills; (h) gills of A. baumannii-infected group showing lamellar fusion (LF) and erythrocyte infiltration (EI); and (i) gills of A. baumannii and octopromycin-treated group showed normal gill structures including gill arches formed by filaments. (H&E), scale bar =12.5 μm.

### MODES OF THE INVENTION

With the rapid emergence and spread of multi-drug resistant super bacteria, there is a demand for the development of novel antimicrobial peptides (AMPs) to deal with serious microbial infections.

The AMPs are promising potential drug candidate materials with broad antibacterial properties. The present inventors prepared a novel peptide “octopromycin” derived from proline-rich protein 5 of Octopus minor and identified its properties. The antibacterial and anti-biofilm effects of octopromycin were confirmed using Acinetobacter baumannii, which is a multi-drug resistant bacterium.

Accordingly, in one aspect, the present invention relates to an antimicrobial peptide having a sequence of 5 or more consecutive amino acids in an amino acid sequence represented by SEQ ID NO: 1:

(SEQ ID NO: 1)
¹RRLIRTDTGPIIYDYFKDQLLKKGMVILRESMKNLKGM³⁸

The AMP according to the present invention may include an amino acid sequence in which one or more amino acid residues are conservatively substituted in a sequence of at least 5, preferably at least 7, more preferably at least 10, most preferably at least 18 consecutive amino acids in the amino acid sequence represented by SEQ ID NO: 1. Conservative amino acid substitutions may include substitutions with amino acid residues that have little or no effect on the size, polarity, charged hydrophobicity, or hydrophilicity of the amino acid residues.

The AMP according to the present invention may have an amino acid sequence represented by SEQ ID NO: 1 or an amino acid sequence having 90% or more, preferably 95% or more, more preferably 98% or more homology with a sequence of 5 or more, preferably 7 or more, more preferably 10 or more, most preferably 18 or more consecutive amino acids in the amino acid sequence represented by SEQ ID NO: 1.

Octopromycin is a peptide having a sequence of 38 amino acids

(¹RRLIRTDTGPIIYDYFKDQLLKKGMVILRESMK
NLKGM³⁸: SEQ ID NO: 1),

and has a net positive charge (+5), a high hydrophobic residue ratio of 36%, and an alpha helical secondary structure. A minimum inhibitory concentration (MIC) and a minimum bactericidal concentration (MBC) for A. baumannii were 50 μg/mL and 200 μg/mL, respectively. In addition, it was confirmed that octopromycin decreased the viability of A. baumannii in a concentration-dependent manner. In addition, through field emission-scanning electron microscopy (FE-SEM) analysis, it was confirmed that octopromycin caused ultrastructural cell wall deformation of A. baumannii to induce resistance by A. baumannii. In addition, it was confirmed by propidium iodide uptake assay that octopromycin at MIC and MBC concentrations significantly penetrated into A. baumannii cells to cause cell membrane loss and apoptosis. In addition, octopromycin treatment increased the production of reactive oxygen species in A. baumannii cells and decreased cell viability of bacteria in a concentration-dependent manner.

In addition, octopromycin exhibited an anti-biofilm effect by not only inhibiting biofilm formation, but also effectively eradiating the existing biofilm formed by A. baumannii.

When Raw 264.7 cell and human embryonic kidney 293T cell lines were treated with octopromycin, it was confirmed that 85% or more of the cells were alive and did not show cytotoxicity to human cells, and safety was confirmed because hemolytic activity was not shown against mouse red blood cells.

In addition, as a result of in vivo zebrafish embryo toxicity studies, octopromycin was not toxic to zebrafish embryos even when treated with 4 μg/mL or more. In addition, it was confirmed that biofilm inhibition and eradication were significantly induced in A. baumannii treated with octopromycin. From the results, it was confirmed that octopromycin was a novel AMP exhibiting antibacterial and antibiofilm effects on multi-drug resistant A. baumannii.

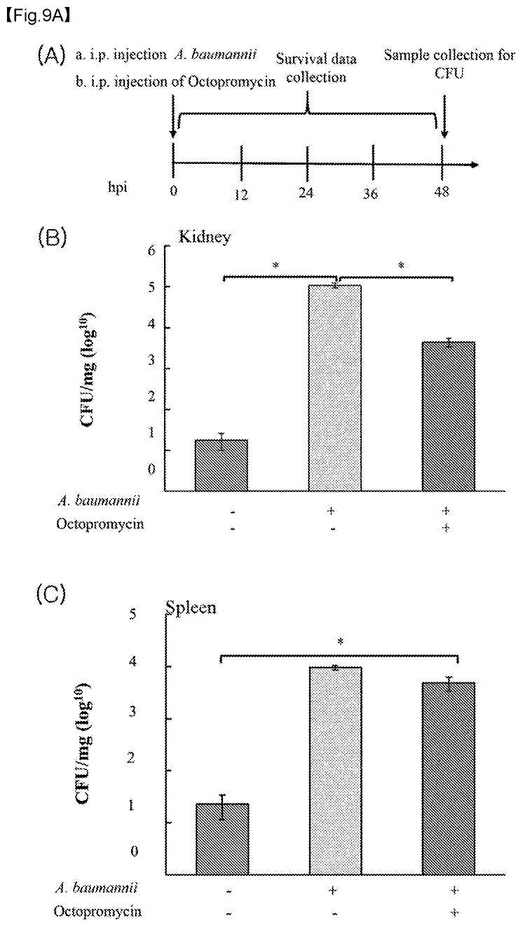

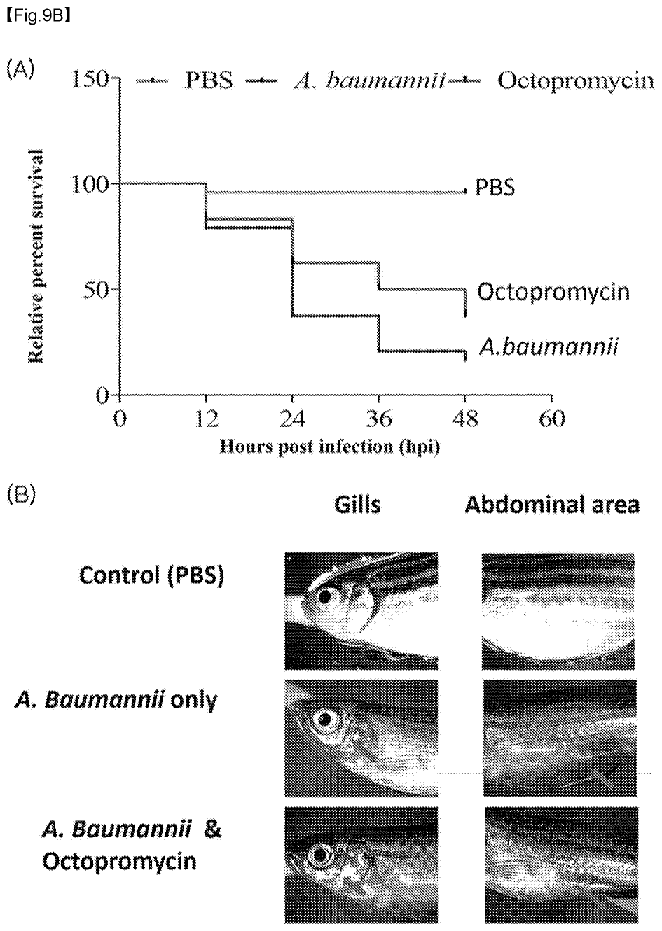

In addition, the effect of treating octopromycin on zebrafish infected with A. baumannii was confirmed. When 2.1×10¹¹ CFU of A. baumannii was injected intraperitoneally in zebrafish, within 48 hours, it was confirmed that the viabilities of an A. baumannii-treated group and an A. baumannii and octopromycin-treated group were 16.67% and 37.5%, respectively, but the viability increased by treatment of octopromycin (FIG. 9D).

In addition, when the number of bacteria (CFU) in the kidneys and spleens of a control group and infected zebrafish were confirmed, bacterial infections were detected in the spleens and kidneys of all experimental groups (FIGS. 9B and 9C), and the CFU was highest in the spleen and kidney of the A. baumannii-infected group, and the second highest in the A. baumannii and octopromycin-treated group. In addition, when the clinical signs of each treatment group were visually confirmed, compared to the PBS-treated group, the A. baumannii-infected group showed skin hemorrhage in the abdominal area and hemorrhage sites which may be caused by blood circulation disorders in the gills and an area surrounded by the gills, but the A. baumannii-infected and octopromycin-treated group showed less hemorrhage in the abdominal area, the gills, and the area surrounded by the gills (FIG. 9E).

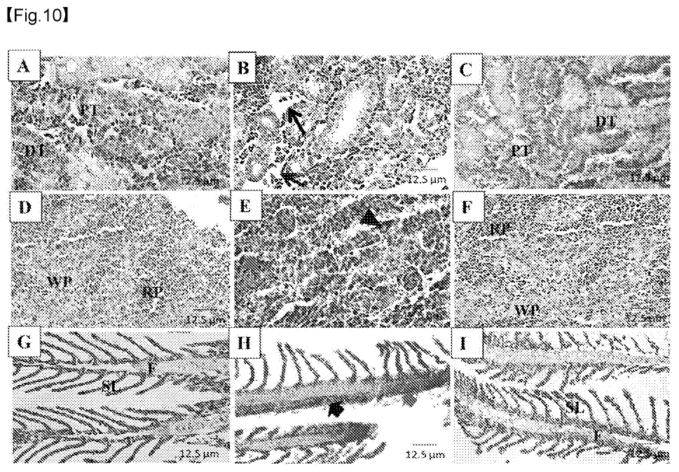

In addition, in another aspect of the present invention, as a result of confirming the effect of octopromycin on A. baumannii-infected zebrafish by histological analysis, compared to the morphological structures of the kidney, spleen, and gills of a normal control group, a zebrafish group infected with A. baumannii observed histological disorders such as degeneration and tissue expansion as the cell space was increased in the kidney tubule (FIG. 10B). Simultaneously, extensive hemorrhage and increased red pulp density were observed in the spleen tissue (FIG. 10E), and lamellar fusion and erythrocyte infiltration were observed in the gills (FIG. 10H). However, in the case of a group treated with octopromycin after A. baumannii infection, tissue disorders and pathological damage were reduced in all tissues (FIGS. 10C, 10F, and 10I). From these results, it was confirmed that octopromycin may be used as a drug capable of controlling A. baumannii.

The AMP of the present invention has antibacterial activity against Acinetobacter sp. bacteria, and particularly, antibacterial activity against Acinetobacter baumannii having multi-drug resistance.

The AMP of the present invention has anti-biofilm formation ability or biofilm eradication ability.

In another aspect, the present invention relates to an antibacterial composition containing the AMP as an active ingredient.

In yet another aspect, the present invention relates to a pharmaceutical composition for preventing or treating Acinetobacter infection containing the AMP as an active ingredient.

The pharmaceutical composition of the present invention is preferably administered orally or parenterally.

In the case of oral administration, the oral administration includes intraoral administration, and the pharmaceutical composition of the present invention is not limited thereto, but may be orally administered in any orally acceptable form including pills, sugarcoated pills, capsules, solutions, gels, syrups, slurries, and suspensions.

In the case of oral tablets, carriers commonly used include lactose and corn starch. A lubricant such as magnesium stearate is also typically added. For oral administration in a capsule form, useful diluents include lactose and dried cornstarch.

When aqueous suspensions are administered orally, the active ingredient is combined with emulsifying and suspending agents. If necessary, sweetening and/or flavoring and/or coloring agents may be added.

A pharmaceutical composition for intraoral administration may be prepared by mixing the active ingredient with a solid excipient and may be prepared in the form of granules for preparation in the form of tablets or sugarcoated pills.

Suitable excipients may use sugar forms such as lactose, sucrose, mannitol and sorbitol, or starch from corn, wheat flour, rice, potato, or other plants, cellulose such as methyl cellulose, hydroxypropylmethyl-cellulose, or sodium carboxymethylcellulose, carbohydrates such as gums including arabic gum and tragacanth gum, or protein fillers such as gelatin and collagen. If necessary, disintegrants or solubilizers in each salt form such as cross-linked polyvinylpyrrolidone, agar and alginic acid or sodium alginate may be added.

As used herein, the term “parenteral” includes subcutaneous, intradermal, intravenous, intramuscular, intraarticular, intrasynovial, intrasternal, intrathecal, intralesional, and intracranial injection or infusion techniques.

The pharmaceutical composition of the present invention may be in the form of a sterile injectable preparation as a sterile injectable aqueous or oil suspension. This suspension may be formulated according to techniques known in the art using suitable dispersing or wetting agents (e.g., Tween 80) and suspending agents. The sterile injectable preparation may also be a sterile injectable solution or suspension (e.g., a solution in 1,3-butanediol) in a non-toxic parenterally acceptable diluent or solvent. Vehicles and solvents that may be acceptably used include mannitol, water, a Ringer's solution, and an isotonic sodium chloride solution. In addition, sterile non-volatile oils are usually used as a solvent or suspending medium. For this purpose, any mild non-volatile oil may also be used by including synthetic mono- or diglycerides. Fatty acids such as oleic acid and its glyceride derivatives are useful in injectable preparations as are pharmaceutically acceptable natural oils (e.g., olive oil or castor oil), especially polyoxyethylated oils thereof.

In a preferred aspect, in the case of parenteral administration, the pharmaceutical composition of the present invention may be prepared as an aqueous solution. Preferably, a physically appropriate buffer solution such as Hank's solution, Ringer's solution, or physically buffered saline may be used. An aqueous injection suspension may be added with a substrate capable of increasing the viscosity of the suspension, such as sodium carboxymethylcellulose, sorbitol, or dextran. In addition, the suspensions of the active ingredient may be prepared as suitable oily injection suspensions. Suitable lipophilic solvents or carriers include fatty acids such as sesame oil or synthetic fatty acid esters such as ethyl oleate, triglycerides, or liposomes.

Polycationic amino polymers may also be used as carriers. Optionally, the suspension may use suitable stabilizers or agents to increase the solubility of the compound and to prepare a highly concentrated solution.

The pharmaceutical composition of the present invention may also be administered in the form of a suppository for rectal administration. These compositions may be prepared by mixing the compound of the present invention with a suitable non-stimulated excipient that is solid at room temperature, but liquid at a rectal temperature. These materials include cocoa butter, beeswax, and polyethylene glycol, but are not limited thereto.

When the pharmaceutical composition of the present invention is applied topically to the skin, the pharmaceutical composition should be formulated in a suitable ointment containing the active ingredient suspended or dissolved in a carrier. Carriers for topical administration of the compound of the present invention include mineral oil, liquid paraffin, white petrolatum, propylene glycol, polyoxyethylene, polyoxypropylene compound, emulsifying wax, and water, but are not limited thereto. Alternatively, the pharmaceutical composition may be formulated as a suitable lotion or cream containing the active compound suspended or dissolved in the carrier. Suitable carriers include mineral oil, sorbitan monostearate, polysorbate 60, cetyl ester wax, cetearyl alcohol, 2-octyldodecanol, benzyl alcohol, and water, but are not limited thereto. The pharmaceutical composition of the present invention may also be topically applied to the lower intestinal tract by rectal suppositories and also with suitable enemas. Topically applied transdermal patches are also included in the present invention.

The pharmaceutical composition of the present invention may be administered by intranasal aerosol or inhalation. Such a composition is prepared according to techniques well known in the field of medicine and may be prepared as a solution in saline using benzyl alcohol or other suitable preservatives, absorption enhancers for increasing bioavailability, fluorocarbons, and/or other solubilizers or dispersants known in the art.

It will be understood that a specific effective amount for a specific patient may vary depending on various factors including the activity of a specific compound used, age, body weight, general health, sex, diet, time of administration, route of administration, rate of excretion, drug combination, and the severity of a specific disease to be prevented or treated.

In yet another aspect, the present invention relates to a method for treating diseases caused by Acinetobacter infection, including administering, to a subject in need thereof, an antimicrobial peptide having a sequence of 5 or more consecutive amino acids in an amino acid sequence represented by SEQ ID NO: 1.

(SEQ ID NO: 1)
¹RRLIRTDTGPIIYDYFKDQLLKKGMVILRESMKNLKGM³⁸.

In the present invention, the term “subject” refers to mammals such as horses, sheep, pigs, goats, and dogs including humans, birds, fish, crustaceans, reptiles, amphibians, etc., which may exhibit a therapeutic effect against Acinetobacter infection, preferably humans.

The peptide may be administered in the form of the pharmaceutical composition described above, and duplicated descriptions will be omitted.

In yet another aspect, the present invention relates to a composition for inhibiting biofilm formation containing the AMP as an active ingredient.

In another aspect, the present invention relates to a gene encoding the AMP.

In yet another aspect, the present invention relates to a recombinant vector containing the gene.

In yet another aspect, the present invention relates to a host cell transformed with the gene or the recombinant vector, and a method for producing an antimicrobial peptide including culturing the host cell in an appropriate medium and culture conditions.

In the present invention, the term “vector” means a DNA construct containing a DNA sequence operably linked to a suitable control sequence capable of expressing DNA in a suitable host. The vector may be a plasmid, a phage particle, or simply, a potential genomic insert. When transformed into an appropriate host, the vector may replicate and function independently of a host genome, or may be integrated into the genome itself in some cases. Since the plasmid is the most commonly used form of the current vector, the “plasmid” and the “vector” are sometimes used interchangeably in the specification of the present invention. However, the present invention includes other forms of vectors that have equivalent functions which have been known or are to be known in the art. Typical expression vectors for expression of a mammalian cell culture are based on, for example, pRK5 (EP 307,247), pSV16B (WO 91/08291), and pVL1392 (Pharmingen).

The term “expression control sequence” refers to a DNA sequence required for the expression of an operably linked coding sequence in a specific host organism. Such a control sequence includes a promoter capable of initiating transcription, any operator sequence for controlling such transcription, a sequence encoding a suitable mRNA ribosome-binding site, and a sequence for controlling the termination of transcription and translation. For example, a control sequence suitable for prokaryotes includes a promoter, any operator sequence, and a ribosome binding site. A control sequence of a eukaryotic cell includes a promoter, a polyadenylation signal, and an enhancer. The factor most influencing the expression level of a gene in a plasmid is a promoter. As a promoter for high expression, a SRa promoter, a cytomegalovirus-derived promoter, and the like are preferably used.

In order to express the DNA sequence of the present invention, any of a wide variety of expression control sequences may be used in the vector. Examples of useful expression control sequences include, for example, early and late promoters of SV40 or adenovirus, a lac system, a trp system, a TAC or TRC system, T3 and T7 promoters, major operator and promoter regions of phage lambda, a control region of a fd code protein, promoters for 3-phosphoglycerate kinase or other glycolytic enzymes, promoters of the phosphatase, for example, Pho5, promoters of a yeast alpha-mating system and other sequences of constitutive and induction known to control the expression of genes of prokaryotic or eukaryotic cells or their viruses, and various combinations thereof. A T7 RNA polymerase promoter 010 may be usefully used for expressing proteins in E. coli.

A nucleic acid is “operably linked” when placed into a functional relationship with another nucleic acid sequence. The nucleic acid may be a gene and control sequence(s) linked in such a method of enabling the gene expression when a suitable molecule (e.g., transcriptional activating protein) binds to the control sequence(s). For example, a pre-sequence or secretory leader peptide is operably linked to DNA; and a promoter or enhancer is operably linked to a coding sequence when affecting transcription of the sequence; or a ribosome binding site is operably linked to a coding sequence when affecting the transcription of the sequence; or the ribosome binding site is operably linked to a coding sequence when positioned to facilitate translation. Generally, “operably linked” means that the linked DNA sequence is contiguous and the secretory leader is also contiguous and present in a reading frame. However, the enhancer does not need to be contiguous. Linkage of these sequences is performed by ligation (linkage) at convenient restriction enzyme sites. When these sites are not present, a synthetic oligonucleotide adapter or linker according to a conventional method is used.

As used herein, the term “expression vector” is a recombinant carrier into which a heterologous DNA fragment is inserted, and generally refers to a double-stranded DNA fragment. Here, the heterologous DNA refers to heteromorphous DNA, which is DNA not found naturally in a host cell. When the expression vector is once present in the host cell, the expression vector may replicate independently of host chromosomal DNA and several copies of the vector and its inserted (heterologous) DNA may be produced.

As well-known in the art, in order to increase the expression level of a transfected gene in the host cell, the corresponding gene needs to be operably linked to transcriptional and translational expression control sequences that exhibit the functions in the selected expression host. Preferably, the expression control sequence and the corresponding gene are included in one expression vector including a bacterial selection marker and a replication origin. When the expression host is a eukaryotic cell, the expression vector needs to further contain an expression marker useful in the eukaryotic expression host.

A wide variety of expression host/vector combinations may be used to express a DNA sequence of a target protein of the present invention. Expression vectors suitable for eukaryotic hosts include expression control sequences derived from, for example, SV40, bovine papillomavirus, adenovirus, adeno-associated virus, cytomegalovirus, and retrovirus. Expression vectors that can be used in bacterial hosts include bacterial plasmids, which may be exemplified by those obtained from E. coli, such as pBluescript, pGEX2T, pUC vector, colE1, pCR1, pBR322, pMB9, and derivatives thereof, plasmids with a wider host range, such as RP4, phage DNA, which may be exemplified by a vary variety of phage lambda derivatives such as λgt10, λgt11, and NM989, and other DNA phages such as M13 and filamentous single-stranded DNA phage. Expression vectors useful for yeast cells are 2μ plasmids and derivatives thereof. A vector useful for insect cells is pVL 941.

A host cell transformed or transfected with the above-described expression vector constitutes yet another aspect of the present invention. As used herein, the term “transformation” means that DNA is introduced into a host to allow DNA to be replicable as an extrachromosomal factor or by chromosomal integration completion. As used herein, the term “transfection” means that an expression vector is accepted by a host cell, whether or not any coding sequence is actually expressed.

The host cell of the present invention may be a prokaryotic or eukaryotic cell. In addition, a host having high DNA introduction efficiency and high expression efficiency of the introduced DNA is usually used. Examples of the host cell to be used include well-known eukaryotic and prokaryotic hosts such as E. coli, Pseudomonas, Bacillus, Streptomyces, fungi, and yeast, insect cells such as Spodoptera frugiperda (SF9), animal cells such as CHO and mouse cells, African green monkey cells such as COS 1, COS 7, BSC 1, BSC 40, and BMT 10, and tissue-cultured human cells, preferably E. coli or Bacillus.

Of course, it should be understood that all of the vectors and expression control sequences do not exhibit functions equally in expressing the DNA sequences of the present invention. Likewise, all of the hosts do not exhibit functions equally for the same expression system. However, those skilled in the art may make appropriate selections among various vectors, expression control sequences, and hosts without departing from the scope of the present invention without undue experimental burden. For example, in selecting a vector, the host needs to be considered, which is because the vector needs to replicate therein. The copy number of the vector, the ability to control the copy number, and expression of other proteins encoded by the vector, such as antibiotic markers, need also to be considered. Even in selecting expression control sequences, various factors need to be considered. For example, relative strength of the sequence, controllability, compatibility with the DNA sequence of the present invention, and the like should be considered, particularly with respect to possible secondary structures. Single-celled hosts need to be selected by considering factors, such as toxicity and secretion characteristics of products encoded by the DNA sequence of the present invention, the ability to correctly fold proteins, culture and fermentation requirements, ease of purifying the products encoded by the DNA sequence of the present invention from the host, and the like. Within these parameters, those skilled in the art may select various vector/expression control sequence/host combinations capable of expressing the DNA sequence of the present invention in fermentation or large-scale animal culture.

Hereinafter, the present invention will be described in more detail through Examples. These Examples are just illustrative of the present invention, and it will be apparent to those skilled in the art that it is not interpreted that the scope of the present invention is limited to these Examples.

### EXAMPLE 1

### Design, Synthesis, and Identification of Properties of Octopromycin

First, as a result of sequencing of the whole body transcript of 0. minor, a praline-rich protein 5 gene was identified. An N-terminal amino acid sequence was used as a template for designing different sizes of AMPs using a prediction tool of the Antimicrobial Peptide Database (APD) (http://aps.unmc.edu/AP) according to criteria of hydrophobicity, net charge, total hydrophobicity ratio, and protein-binding capacity.

The designed peptides were synthesized using a solid-phase peptide synthesis technique (AnyGen Co., Korea) and purified by reverse-phase HPLC using a SHIMADZU C18 analytical column (Shimadzu HPLC LabSolutions, Japan). After preliminary screening to confirm the antibacterial activity, a peptide consisting of 38 amino acids (¹RRLIRTDTGPIIYDYFKDQLLKKGMVILRESMKNLKGM³⁸; SEQ ID NO: 1) was selected and named octopromycin.

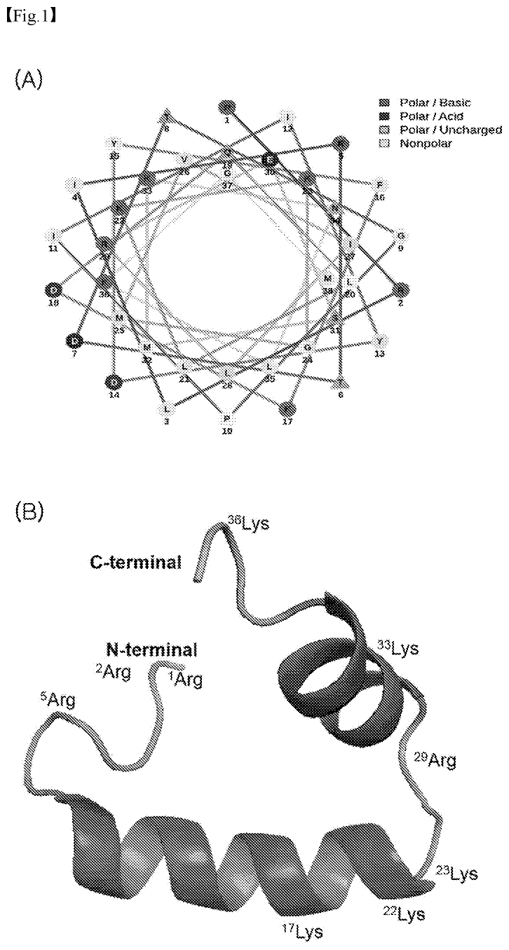

The octopromycin exhibited a net positive charge +5 due to relatively highly positively charged amino acid residues of 13% lysine (K) and 10% arginine (R), and exhibited physicochemical properties with a high hydrophobicity ratio (36%) due to the presence of 13% leucine (L), 10% isoleucine (I), and 7% methionine (M). In addition, it was predicted that 9 hydrophobic residues were present on the same surface of the octopromycin structure. In addition, the octopromycin had a molecular mass of 4542.495 Da and a protein binding potential (Boman index) of 2.11 kcal/mol. Based on a Wimley-White total residue hydrophobicity scale, the octopromycin had a hydrophobicity of 8.12 kcal/mol. Based on comparative alignment of known AMP and octopromycin sequences, the most similar to a Rugosin-LK2 peptide (33 aa) having +4 net charge and 42% hydrophobicity ratio was identified. It was confirmed that in secondary and tertiary predicted structures of the octopromycin, the octopromycin had an alpha-helical structure (FIGS. 1A and 1B).

### EXAMPLE 2

### Identification of MIC and MBC of Octopromycin in A. baumannii

In Example 2, in order to confirm the antibacterial effect of octopromycin on A. baumannii bacteria having multi-drug resistance, a minimum inhibitory concentration (MIC) and a minimum bactericidal concentration (MBC) of octopromycin were confirmed.

The MIC and MBC levels of octopromycin in A. baumannii were determined using media dilution tests and subcultures, respectively, according to the guidelines of the Clinical and Laboratory Standards Institute (CLSI, 2008 M27-A3). Several concentrations of octopromycin (0, 5, 10, 25, 50, 100, and 200 μg/mL) were tested as standard antibiotics (positive control) together with chloramphenicol (10 μg/mL, Sigma-Aldrich, USA) acting on A. baumannii growth and the MBC of chloramphenicol was confirmed according to the same procedure. The MIC was determined as the minimum concentration required to inhibit visual growth of A. baumannii. The MBC was measured by subculturing 10 μL of a medium collected from a dilution series, which showed no visible growth after 24 hours of culture on a TSA medium plate, and the lowest concentration that did not produce colonies after 24 hours of growth on the TSA medium. For analysis of time-kill kinetics, A. baumannii was cultured with octopromycin at various concentrations (0, 25, 50, 100, and 200 μg/mL) in the TSA medium at 25° C., and at different time intervals (0, 3, 6, 9, 12, 18, and 21 h), the absorbance was measured at 595 nm using a microplate reader (Bio-Rad, Saint Louis, USA) to confirm an inhibitory effect after culturing.

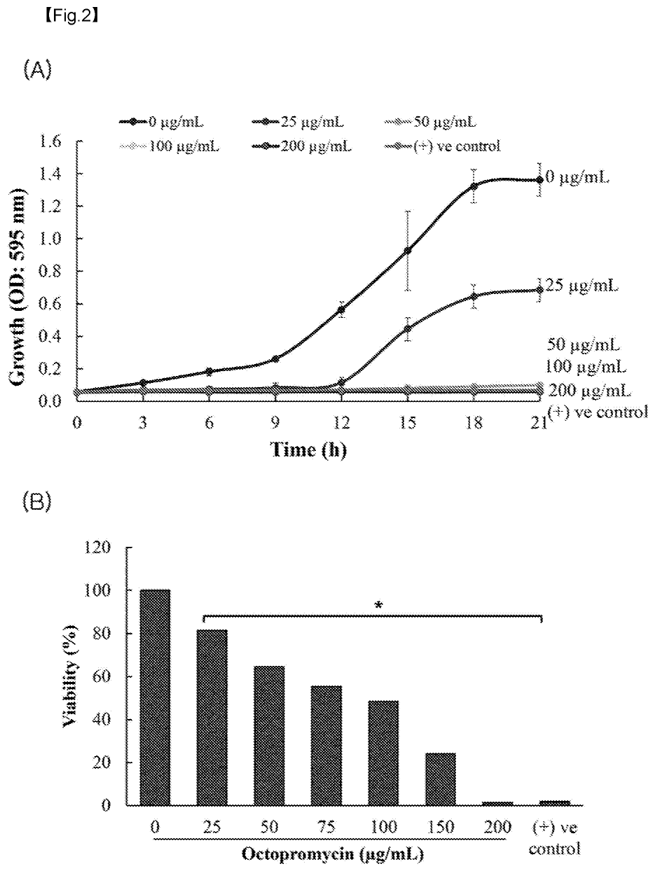

As a result, it was confirmed that the MIC of octopromycin was 50 μg/mL and the MBC was 200 μg/mL, and the octopromycin showed a clear antibacterial effect on A. baumannii. In addition, as a result of time-kill kinetics analysis, clear growth inhibition against A. baumannii was shown even at a MIC concentration or less (FIG. 2A). When chloramphenicol (positive control) was treated at a concentration of 10 μg/mL, growth inhibition was similar to that of 50 μg/mL of octopromycin. In addition, when octopromycin was treated at a low concentration of 25 μg/mL compared to A. baumannii not treated with octopromycin, partial growth inhibition was shown for A. baumannii up to 12 hours. To further evaluate the growth inhibition in A. baumannii after octopromycin treatment, cell viability was measured using the MTT assay.

A. baumannii was cultured in TSB (OD-0.25, 2.5 x 106 CFU/mL) and treated with octopromycin at various concentrations (0, 25, 50, 75, 100, 150, and 200 μg/mL). After culturing for 24 hours in a shaking incubator at 180 rpm at 25° C., A. baumannii was centrifuged at 3500 rpm for 10 minutes, and the cells were washed with PBS. For cell viability assay, A. baumannii cells reacted with 20 μL of an MTT reagent (Sigma Aldrich, USA) for 30 minutes, and the samples were resuspended in dimethylsulfoxide (Sigma Aldrich, USA). The cell viability was measured based on OD at 570 nm using a microplate reader (Bio-Rad, Saint Louis, USA).

As a result, the cell viability was significantly reduced in octopromycin-treated A. baumannii in a concentration-dependent manner (0 to 200 μg/mL) compared to an untreated control group (p <.05) (FIG. 2B). It was confirmed that the lowest cell viability of A. baumannii treated with 200 μg/mL of octopromycin was 1.54%.

### EXAMPLE 3

### Morphological Change Analysis in A. baumannii by Octopromycin Treatment

To understand a mode of action of octopromycin, FE-SEM analysis was performed at MIC and MBC concentrations to estimate the ultrastructure changes of A. baumannii.

First, A. baumannii cells were treated with octopromycin at MIC (50 μg/mL) and MBC (200 μg/mL) for 9 hours. As a negative control group, an untreated A. baumannii culture solution was used, and all other steps were performed identically for the sample. Subsequently, the culture solution was centrifuged at 3500 rpm for minutes and the pellet was washed with phosphate saline buffer PBS and prefixed with 2.5% glutaraldehyde for 30 minutes. Then, the prefixed cells were washed with PBS and continuously dehydrated using ethanol (30, 50, 70, 80, 90, and 100%). The fixed cells were dried and coated with platinum using an ion sputter (E-1030, Hitachi, Japan). The treated cells and the control cells were observed with FE-SEM (Sirion FEI, Netherlands).

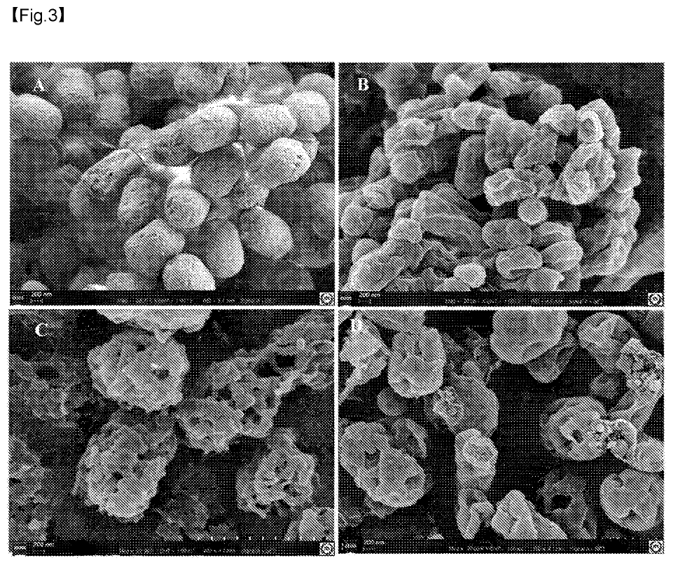

As a result of SEM analysis, the destruction of an A. baumannii membrane was evident under octopromycin treatment at MIC and MBC (FIG. 3), but was not shown in untreated cells. As expected, untreated A. baumannii had no pores on the cell surface, and was relatively less destroyed and less damaged (FIG. 3A). In contrast, in A. baumannii treated with octopromycin at MIC (FIG. 3B) and A. baumannii treated with octopromycin at MBC (FIG. 3C), severe membrane destruction such as rough membrane appearance, transparent pores, and irregularly shaped cells was clearly observed. It was also confirmed that octopromycin induced severe morphological and ultrastructural changes at the MBC level than the MIC and positive control groups (FIG. 3D).

### EXAMPLE 4

### Effect of Octopromycin on Changes in Membrane Permeability of A. baumannii

Propidium iodide (PI) uptake assay was performed to evaluate an effect of octopromycin on association with the membrane permeability and apoptosis of A. baumannii.

The PI was a red fluorescent nuclear and chromosome counter dye that indicated changes in cell membrane permeability, and was used to detect the presence of dead cells and simultaneously observe live cells by observing green fluorescence (FDA). A. baumannii treated with octopromycin at MIC; 50 μg/mL and MBC; 200 μg/mL levels and control cell suspensions were centrifuged (5000 rpm for 2 minutes), washed with PBS and the pellets were suspended in PBS. Subsequently, the cells were cultured with 20 μg/mL of PI (Sigma Aldrich, USA) and 8 μg/mL of FDA (Sigma Aldrich, USA) at 25° C. for 30 minutes in the dark, and finally, the remaining dye was washed with PBS. Finally, a 5 μL drop of each suspension was placed on a cover slip and fluorescence images were observed using a CLSM integrated with a scan head on an Axiovert 200M inverted microscope (Carl Zeiss, Germany).

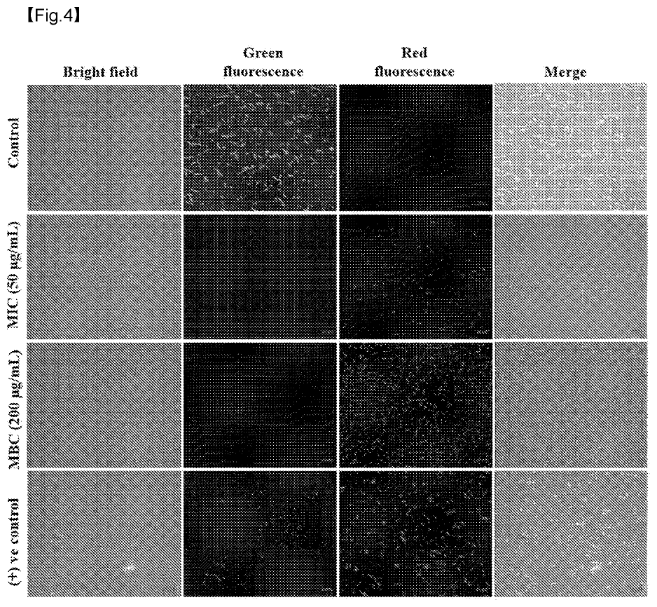

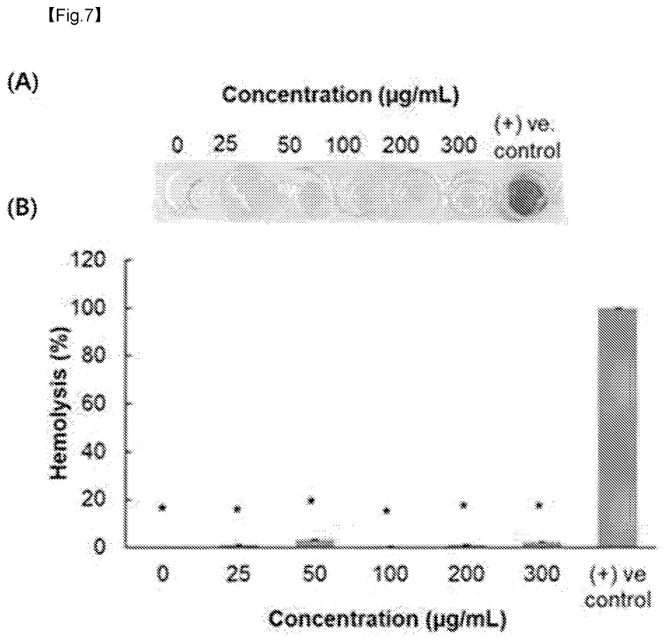

As a result, it was confirmed that significant cell membrane damage and destruction of A. baumannii were induced in a concentration-dependent manner by octopromycin treatment (FIG. 4). In untreated A. baumannii, there were no PI-stained red fluorescent cells (FIG. 4C), but FDA-stained green fluorescent cells were all shown (FIG. 4B). In contrast, at the MIC level, octopromycin-treated A. baumannii had more PI-stained red fluorescent cells (FIG. 4G) and fewer FDA (green fluorescent)-stained cells (FIG. 4F). In addition, almost all A. baumannii cells showed red fluorescence signals even when treated with MBC-level octopromycin (FIG. 4K), and a chloramphenicol-treated positive control group (FIG. 7O) also showed high membrane permeability (red fluorescence).

### EXAMPLE 5

### Effect of Octopromycin on ROS Production in A. baumannii

To further examine a mode of action of octopromycin related to endogenous ROS production in A. baumannii by octopromycin, H2DCF-DA assay was performed.

The accumulation of ROS in A. baumannii was quantified using fluorescent probe carboxy-H2DCF-DA (Invitrogen, USA). An A. baumannii culture solution (OD-0.25, 5×10⁶ CFU/mL) was treated with octopromycin (MIC; 50 μg/mL and MBC; 200 μg/mL) and cultured at 25° C. for 10 hours, and then, cells were harvested by centrifugation at 5000 rpm for 2 minutes. To detect ROS levels, the cells were stained with H2DCF-DA (100 μg/mL) and cultured at room temperature (24±1° C.) for 30 minutes and then centrifuged at 5000 rpm for 2 minutes. The cells were washed with ×1 PBS and dichloro-fluorescein (DCF) fluorescence was measured using CLSM (LSMS Live Configuration Variotwo VRGB, Zeiss, Germany) at an excitation wavelength of 488 nm and an emission wavelength of 535 nm.

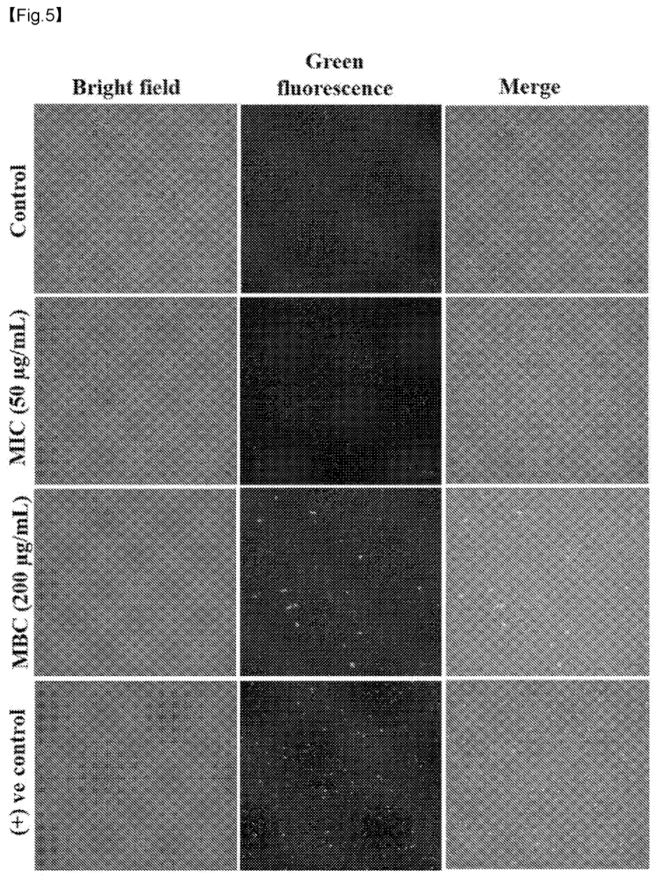

As a result, it can be seen that the ROS level induced in octopromycin-treated A. baumannii increased in a concentration-dependent manner (FIG. 5). That is, a large amount of A. baumannii cells were stained with green fluorescence at a MBC (200 μg/mL) rather than MIC (50 μg/mL) of octopromycin. This indicates that octopromycin may reduce viable cells of A. baumannii by excessively increasing the ROS level to induce oxidative stress.

### EXAMPLE 6

### Effect of Octopromycin on Biofilm Formation Inhibition and Eradication of A. baumannii

In order to investigate an effect of octopromycin on the biofilm of A. baumannii, analysis for biofilm formation inhibition and eradication was performed.

To examine the inhibitory effect of an octopromycin peptide on biofilm formation, A. baumannii was cultured in TSB. The bacteria were diluted to 5×10⁵ CFU/mL in a TSB medium, then 97.5 mL and 90 mL of bacterial suspensions were mixed with 2.5 mL and 10 mL of peptides, respectively, to reach 50 μg/mL (MIC) and 200 μg/mL (MBC), and cultured on a 96-well plate for 24 hours. Then, the supernatant was carefully removed, and the formed biofilm was fixed with 100% methanol for 10 minutes. After methanol was removed, the biofilm was stained with 0.1% crystal violet for 30 minutes. Subsequently, the plate was washed 3 times with distilled water. Finally, the biofilm was completely dissolved in 95% ethanol and absorbance was measured at 495 nm using a microplate reader (Bio-Rad, USA).

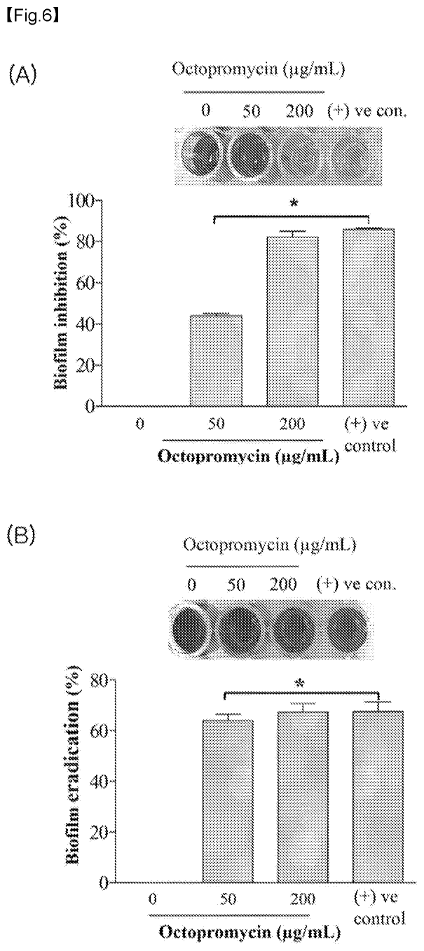

As a result, A. baumannii treated with octopromycin significantly inhibited the biofilm formation in a concentration-dependent manner at 24 hours after treatment (FIG. 6). Relative inhibition of biofilm formation of 44.0% and 82.3% was confirmed based on crystal violet staining levels at MIC and MBC compared to the untreated control, respectively (FIG. 6A). In particular, octopromycin treatment at the MBC concentration showed an almost similar formation inhibitory effect to that of standard antibiotics by confirming 85.9% biofilm formation inhibition after treating chloramphenicol (10 μg/mL) as a positive control group. These data show that octopromycin has strong anti-biofilm activity against multi-drug resistant A. baumannii.

Next, the effect of octopromycin on biofilm eradication in A. baumannii was confirmed. To measure the eradication effect of the already formed biofilm, 100 μL of A. baumannii (5×10⁵ CFU/mL) was cultured in TSB for 24 hours. The culture medium was removed and the wells were carefully washed with PBS to eradicate planktonic bacteria. The wells were added with octopromycin (50 μg/mL (MIC) and 200 μg/mL (MBC)) and further cultured for 24 hours. The biofilm was stained with crystal violet for 30 minutes, washed three times with PBS, and dissolved in 95% ethanol, and the absorbance was measured at 495 nm using a microplate reader (Bio-Rad, USA).

Crystal violet staining results clearly showed the biofilm eradication effect of octopromycin (FIG. 6B). Significant reduction (p <.05) in biofilm was confirmed as 64.0% and 64.2% at MIC and MBC levels, respectively, at 24 hours after treatment compared to untreated A. baumannii (100%).

Overall results suggest that octopromycin not only inhibits biofilm formation, but also effectively eradicates existing biofilms formed by A. baumannii.

### EXAMPLE 7

### Effect of Octopromycin on Hemolytic Activity

Systemic application of octopromycin as an antibiotic requires low toxicity to red blood cells. Accordingly, an in vitro hemolysis assay was performed using mouse red blood cells as a positive control group and Triton X-100.

The hemolytic activity of the peptide was evaluated using mouse red blood cells (RBC). The RBCs were washed 3 times with phosphate buffered saline (PBS) until the supernatant was clear. Different concentrations of octopromycin (0, 25, 50, 100, 200, 300 μg/mL) were added to 1.5 ml Eppendorf tubes. The RBCs were added at a final concentration of 8% (v/v) in a total volume of 500 μL. The RBCs were incubated for 1 hour, and then centrifuged for 10 minutes and the absorbance of the supernatant was measured at 414 nm. The RBCs suspended in PBS and 1% Triton X-100 showed zero and 100% hemolysis, respectively.

Percent hemolysis was calculated using Equation:

Hemolysis (%)=(A₄₁₄ of Peptide−A₄₁₄ of PBS)/(A₄₁₄ of Triton−A₄₁₄ of PBS)×100

The lowest and highest hemolytic activities were found in untreated red blood cells (0%) and Triton X-100 (100%)-treated red blood cells (FIGS. 7A and 7B). In contrast, octopromycin-treated red blood cells showed a significantly low hemolytic effect (p≤0.05) at various experimental concentrations (25, 50, 100, 200, and 300 μg/mL). The results of these hemolytic effects indicated that octopromycin was not involved in lysis of the membrane lipid bilayer of red blood cells and was not cytotoxic to mammalian cells even when treated at MBC (200 μg/mL) or higher.

### EXAMPLE 8

### In Vitro and In Vivo Toxicity Analysis of Octopromycin

To demonstrate the potential safety of octopromycin for therapeutic applications, in vitro and in vivo toxic effects were measured through the MTT assay using two types of mammalian cells HEK293 and Raw 264.7 and zebrafish embryos (by mortality and teratogenicity estimation).

To measure the cytotoxicity of octopromycin on HEK293T (American Type Culture Collection ATCC-11268) and Raw 264.7 cells, the cells were treated with octopromycin and cell viability was evaluated. The cells were harvested, counted and seeded at a density of 2.5×10⁶ cells per well in a 96-well plate and cultured for 24 hours at 37° C. and 5% CO₂. The cells were treated with different concentrations of octopromycin (0 to 800 μg/mL) and cultured for 24 hours. After culturing, the culture medium was replaced with a fresh medium, and 10 μL of MTT (5 mg/mL) was added to each well and incubated in a CO₂ incubator for 4 hours. The formed formazan crystals were dissolved in 50 μL of DMSO and absorbance was measured at 570 nm using a microplate reader (Bio-Rad Laboratories, Inc, USA). Cells treated with the medium (control) were considered as 100% viability (control group), and current viability was normalized and calculated for the untreated control group.

Wild-type (AB) adult zebrafish was maintained in an automated water circulation system at 28° C. under a photoperiod of 14 h (light): 10 h (dark). After zebrafish (to 4 months of age) was mated, embryos were collected at a 1 hours post fertilization (1 hpf) stage and rinsed with embryo water. First, the viability of newly fertilized embryos was identified under a stereoscopic microscope (Nikon, SMZ1000, Japan) before use in experiments. To determine toxicity levels at 24, 48, 72, and 96 hours after exposure, embryos at stages 1-2 hpf were exposed to various concentrations of octopromycin (0 to 8 μg/mL) for 96 hours.

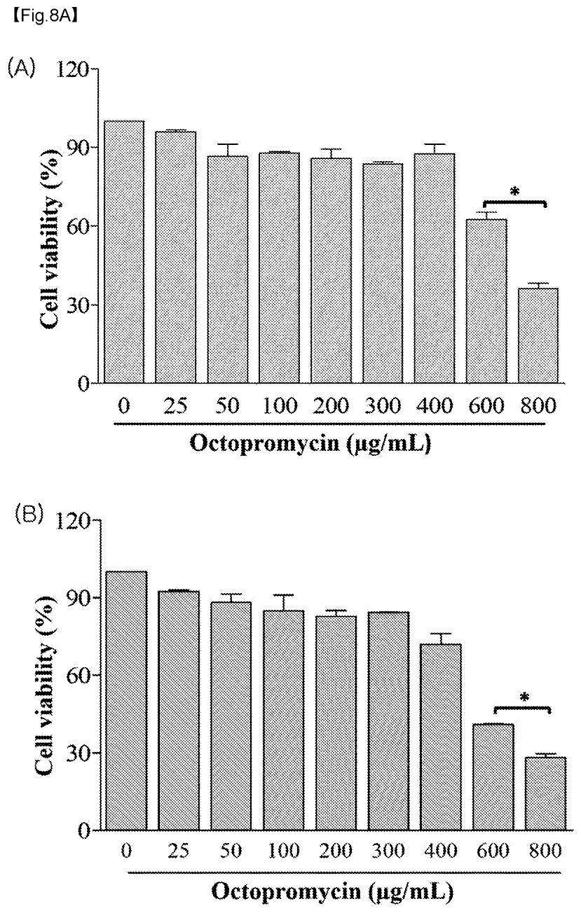

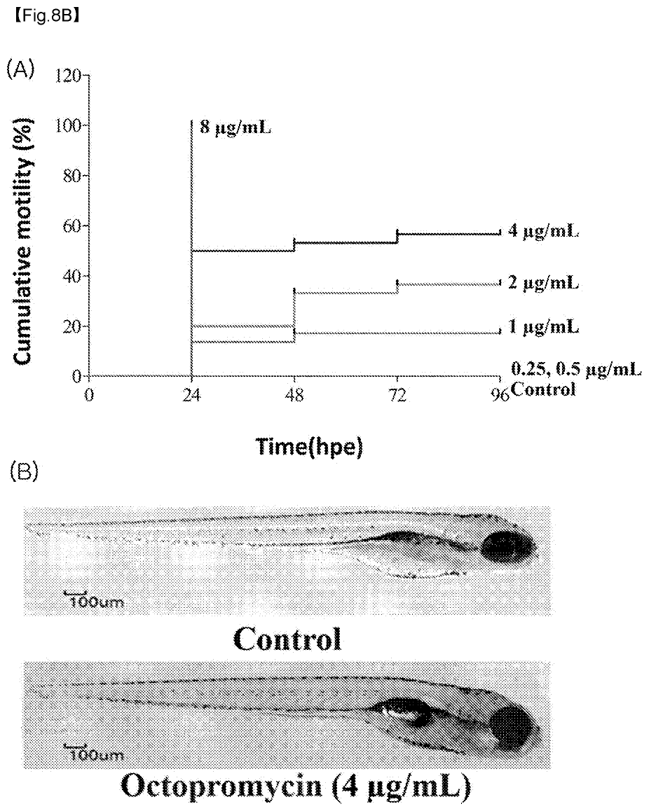

As a result, there was no specific morphological change in octopromycin-treated HEK293 and Raw 264.7 cells up to a maximum concentration of 400 μg/mL ((A) and (B) in FIG. 8A), and the viabilities of HEK293 cells and Raw 264.7 cells were 85% and 80%, respectively. However, at higher concentrations (>400 μg/mL), the cell viability was significantly reduced in both types. Overall results showed that octopromycin had no toxic effect on both HEK293 and Raw 264.7 cells at the MBC level (200 μg/mL), which showed the potential usability in in vivo clinical trials.

In addition, to evaluate in vivo toxicity, the cumulative mortality of developing zebrafish embryos for 96 hours was calculated at octopromycin exposure (0, 0.25, 0.50, 1.00, 2.00, 4.00, and 8.00 μg/mL). There was no embryo mortality in unexposed embryos and octopromycin at a low concentration (0.25 μg/mL) ((A) in FIG. 8B). However, concentration-dependent mortality patterns occurred by an increase in octopromycin concentration. 100% mortality occurred in embryos exposed for 24 hpe to 8.0 μg/mL of octopromycin, but compared to a control group, no abnormal appearance of embryos was observed at a maximum non-lethal concentration of 4 μg/mL ((B) in FIG. 8B).

### EXAMPLE 9: Confirmation of effect of octopromycin on A. baumannii-infected animal model

The effect of octopromycin as an antibacterial agent was confirmed using zebrafish as an animal model.

A total of 72 zebrafish (average weight: 0.35±0.05 g) were divided into 3 groups: (1) non-infected control group treated with PBS and water, (2) A. baumannii-infected group, and (3) A. baumannii-infected and octopromycin-treated group (octopromycin-treated group).

Zebrafish was anesthetized using system water containing 160 μg/mL of tricaine (ethyl 3-aminobenzoate methane sulfonate). The control group ((1) group) was injected with 10 μL of nucleous free water (NFW) and 20 μL of PBS using a Hamilton® syringe. The A. baumannii-infected group ((2) group) was injected intraperitoneally with 10 μL of NFW, and the octopromycin-treated group ((3) group) was injected intraperitoneally with 10 μL of octopromycin (10 μg/fish). Then, A. baumannii was injected intraperitoneally (0 hpi) in the A. baumannii-infected group and the octopromycin-treated group at a dose of 20 μ1 (3.4×10¹¹ CFU) per fish, and then injected every 2 hpi until 48 hpi. To confirm the antibacterial effect of octopromycin, fish mortality was recorded every 12 hpi and the viability of each group was examined.

Zebrafish injected with PBS (control group) survived (95.83%) except for one individual, and the viability of a group injected with A. baumannii and octopromycin was 37.5% at 48 hours. Meanwhile, the A. baumannii-infected group had the viability of 16.67% within 48 hours ((A) in FIG. 9B).

In addition, the number of bacteria (CFU) in the kidneys and spleens of the control group and the infected zebrafish was observed. 3 fish from each group at the 80% mortality stage of the A. baumannii-infected group was selected, and then the spleen and the kidney were collected, and individual spleen and kidney tissues from each zebrafish were homogenized in PBS and the homogenates were serially diluted and loaded onto an agar plate (TSA) to measure A. baumannii CFU.

Bacterial infection was detected in the spleen and kidney of all experimental groups ((B) and (C) in FIG. 9A). The number of bacteria was highest in the spleen and kidney of the A. baumannii-infected group, followed by the A. baumannii and octopromycin-treated group. Compared to the two groups, the control group had the lowest CFU in both tissues. As shown in (B) of FIG. 9B, the A. baumannii-infected group showed distinct external clinical signs compared to the PBS-treated group. The skin hemorrhage in the abdominal area and hemorrhage sites caused by impaired blood circulation in the gills and in the area surrounded by the gills may be shown.

Interestingly, however, the A. baumannii-infected and octopromycin-treated group showed less hemorrhage in the abdominal area, the gills, and the area surrounded by the gills. Collectively, these results suggest that octopromycin may be used as an antimicrobial agent against A. baumannii which is a multidrug-resistant bacterium.

### EXAMPLE 10

### Confirmation of Effect of Octopromycin on A. baumannii-Infected Zebrafish Through Histological Analysis

Two zebrafish were selected from each group at the 80% death stage (48 hpe) and the spleen, gills and kidneys were collected separately. Structural changes in A. baumannii-infected zebrafish treated with octopromycin were confirmed by histological analysis. Briefly, two fish from each group (control group, A. baumannii-infected group and octopromycin-treated group) were anesthetized with excess tricaine and the spleen, gills, and kidneys were surgically removed. Each tissue was immersed in PBS and fixed in 10% neutral buffered formalin. After a 12-hour washing step, tissues were dehydrated using a graded series of alcohols in a semi-closed benchtop tissue processor (Leica® TP1020, Germany). The dehydrated tissues were paraffin-embedded (Leica®EG1150 Tissue Embedding Center, Germany), sectioned at 4 (Leica®RM2125 microtome, Germany), and stained with Hematoxylin and Eosin (H & E) (Sigma, Aldrich). The stained tissues of each group were imaged using a digital camera (LEICA®DCF450-C, Germany) connected to a microscope (LEICA®DM 3000 LED).

As a result, as shown in FIG. 10, the morphological structures of the kidneys, spleen, and gills of the control group were normal as shown in FIGS. 10A, 10D, and 10G. In the A. baumannii-infected group, histological disorders such as degeneration and tissue expansion were observed as a cell space was increased in the kidney tubule (FIG. 10B). At the same time, extensive hemorrhage and increased red pulp density were observed in the spleen tissue (FIG. 10E), and lamellar fusion and erythrocyte infiltration were observed in the gills (FIG. 10H). In contrast, in all tissues of the group infected with A. baumannii and then treated with octopromycin, tissue disorders and pathological damage were reduced overall. The kidneys showed undamaged tubules and few cell spaces (FIG. 10C), the spleen had well-distributed red and white pulps, and had no hemorrhage (FIG. 10F), and the gills had a normal structure (FIG. 10I). From the in vivo results, it was confirmed that octopromycin may be used as a drug capable of controlling A. baumannii.

As described above, specific parts of the present invention have been described in detail, and it will be apparent to those skilled in the art that these specific techniques are merely preferred embodiments, and the scope of the present invention is not limited thereto. That is, the substantial scope of the present invention is defined by the appended claims and their equivalents.

1. An antimicrobial peptide having a sequence of 5 or more consecutive amino acids in an amino acid sequence represented by SEQ ID NO: 1:
(SEQ ID NO: 1)
¹RRLIRTDTGPIIYDYFKDQLLKKGMVILRESMKNLKGM³⁸.

2. The antimicrobial peptide of claim 1, wherein the antimicrobial peptide has a sequence of 18 to 23 consecutive amino acids in the amino acid sequence represented by SEQ ID NO: 1.

3. The antimicrobial peptide of claim 1, wherein the antimicrobial peptide has antibacterial activity against Acinetobacter sp. bacteria.

4. The antimicrobial peptide of claim 3, wherein the antimicrobial peptide has antibacterial activity against Acinetobacter baumannii.

5. The antimicrobial peptide of claim 1, wherein the antimicrobial peptide has biofilm anti-formation ability or biofilm eradication ability.

60. An antibacterial composition comprising the antimicrobial peptide of claim 1 or 2 as an active ingredient.

7. A pharmaceutical composition for preventing or treating Acinetobacter infection comprising the antimicrobial peptide of claim 1 or 2 as an active ingredient.

8. A method for treating diseases caused by Acinetobacter infection, comprising administering, to a subject in need thereof, an antimicrobial peptide having a sequence of 5 or more consecutive amino acids in an amino acid sequence represented by SEQ ID NO: 1:
(SEQ ID NO: 1)
¹RRLIRTDTGPIIYDYFKDQLLKKGMVILRESMKNLKGM³⁸.

9. The method for treating diseases caused by Acinetobacter infection of claim 8, wherein the antimicrobial peptide has a sequence of 18 to 23 consecutive amino acids in the amino acid sequence represented by SEQ ID NO: 1.

10. A composition for inhibiting biofilm formation comprising the antimicrobial peptide of claim 1 or 2 as an active ingredient.

11. A gene encoding the antimicrobial peptide of claim 1 or 2.

12. A recombinant vector comprising the gene of claim 11.

13. A host cell transformed with the recombinant vector of claim 12.

14. A method for producing an antimicrobial peptide by culturing the host cell according to claim 13.


SEQUENCE LISTING (1 sequence)

SEQ ID NO: 1 - protein, 38 aa, Artificial Sequence
  Octopromycin
RRLIRTDTGPIIYDYFKDQLLKKGMVILRESMKNLKGM